(19) United States
(12) Patent Application Publication
(10) Pub. No.: US 2007/0168040 A1
(43) Pub. Date: Jul. 19, 2007

(54) Motion disc spike seating instrument

(76) Inventor: Douglas Raymond, Randolph, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/333,588

(22) Filed: Jan. 17, 2006

(51) Int. Cl. A61F 2/44; A61F 2/46; A61B 17/88

(52) U.S. Cl. 623/17.15; 606/99

(57) ABSTRACT

A seating device having a shaft and a U-shaped head for interposition between the prosthetic endplates of an intervertebral motion disc which, upon pivoting, more fully seats the teeth of the prosthetic endplates that lie upon the outer surfaces of the endplates.

### BACKGROUND OF THE INVENTION

The leading cause of lower back pain arises from rupture or degeneration of lumbar intervertebral discs. Pain in the lower extremities is caused by the compression of spinal nerve roots by a bulging disc, while lower back pain is caused by collapse of the disc and by the adverse effects of articulation weight through a damaged, unstable vertebral joint. One proposed method of managing these problems is to remove the problematic disc and replace it with a prosthetic disc that allows for the natural motion between the adjacent vertebrae (“a motion disc”).

Surgeons currently desire an instrument that will accurately and consistently seat a motion disc within a disc space. There are several qualities to a properly seated disc. When the teeth on the outer surfaces of the inserted disc are fully engaged, there is a significantly reduced risk that the motion device will shift after surgery, which could result in expulsion of the prosthesis.

The current state of the art in motion discs typically involves the use of a spreader device that is inserted between and pressed against the opposing prosthetic disc endplates. Upon mechanical actuation, the spreader device opens and the prosthetic endplates are spread apart. One problem with these spreader devices is that they are often very cumbersome. The spreader devices also provide the surgeon with little tactile feel, which assists the surgeon in determining whether the device has been properly seated. Lastly, there is limited space for fitting a spreading mechanism, especially, in the cervical spine.

In some instances, surgeons apply a perpendicular force to the prosthetic vertebral endplates by use of distraction pins.

US Published Patent Application No. 2004/0167537 (“Errico”) discloses an instrument called a leveler 600 that has a tuning fork shape and is adapted to be inserted between the endplates of an articulating intervertebral motion disc. According to Errico, this leveler functions as a wedge to force the endplates apart and secure purchase of the stabilizing spikes. However, the use of a wedge between the endplates is problematic in that it typically requires high impaction forces in order to properly locate itself deep within the disc space. These high impaction force may undesirably jostle the motion disc, and may also cause undesirable posterior movement of the endplate (and core, if present) components.

Therefore, current seating techniques can not provide both tactile feedback and visual confirmation, and require additional fluoroscopic imaging to confirm the appropriateness of the seating.

### SUMMARY OF THE INVENTION

It is an object of the present invention to ensure that the teeth on the outer surface of the prosthetic endplates are fully engaged with the bony vertebral endplates without having high impaction forces.

The invention comprises a tuning fork-like seating device having a proximal shaft, an intermediate portion, and distal tynes extending from the intermediate portion. The seating device has a head portion that is relatively thin in relation to the distance between the endplates, so that when it is inserted between the prosthetic endplates that are at least partially engaging their respective bony endplates, only one of the upper and lower surfaces of the tynes contacts an inner surface of the prosthetic endplates. By moving the proximal shaft either superiorly or inferiorly, the device will pivot about its intermediate portion upon a proximal portion of a first endplate, and the distal tynes will bear against the distal inner surface of the second prosthetic endplate, thereby causing the prosthetic endplates to separate from each other and pushing the exterior teeth into the bony vertebral endplates. This process can then be repeated by moving the proximal portion of the device in the opposite direction, so that the device will pivot about its intermediate portion upon a proximal portion of the second endplate, and the distal tynes will bear against the distal inner surface of the first prosthetic endplate,

The slim nature of the design of the present invention not only greatly improves the instrument's tactile feel and enables visual confirmation of the proper placement of the prosthetic components, it also does so with requiring high impaction forces.

Therefore, in accordance with the present invention, there is provided an assembly for seating prosthetic endplates of an intervertebral disc between upper and lower vertebral endplates, comprising:
a) an upper prosthetic endplate having an outer surface having teeth for engaging the upper vertebral endplate and an inner surface having a peripheral rim,
b) a lower prosthetic endplate having an outer surface having teeth for engaging the lower vertebral endplate and an inner surface having a peripheral rim,
   c) a seating device having a proximal shaft, an intermediate portion, and first and second distal tynes extending distally from the intermediate portion, each tyne having a distal end, an upper surface and a lower surface, wherein the inner surfaces of the endplates oppose each other to define a gap there-between,
   wherein the intermediate portion and the tynes of the seating device are positioned within the gap, and
   wherein the thickness of the tynes is less than the thickness of the gap so that the distal end of one tyne does not contact an endplate.

### DESCRIPTION OF THE FIGURES

FIG. 1 discloses a perspective view of a first embodiment of the seating device of the present invention.

FIG. 2 discloses a perspective view of the seating device of the present invention fully inserted between a pair of prosthetic endplates.

FIGS. 3a and 3b disclose perspective and side views of a second embodiment of the seating device of the present invention, wherein the upper and lower surfaces of the tynes are concave.

FIG. 4 is a perspective view of an embodiment wherein the proximal shaft has an offset portion.

FIG. 5 shows the device having the offset shaft interposed between a pair of distractor tubes.

FIG. 6 is a perspective view of an embodiment wherein the proximal shaft has a docking arm extending therefrom.

FIG. 7 is a perspective view of an embodiment having a docking arm, wherein both the head portion and the docking arm are individually pivotally connected to a pair of handles.

FIG. 8 is an embodiment having one short and one long curved tyne.

### DETAILED DESCRIPTION OF THE INVENTION

In some embodiments, the invention includes a proximal shaft and distally extending tynes. In some embodiments, there is provided an intermediate section that extends transverse to the proximal shaft, and from which the distal tynes extend distally. In this manner, the intermediate and tynes portions comprise a head portion, and the device is shaped substantially similar to a tuning fork. The head is inserted between the prosthetic endplates, with one tyne being inserted on either side of the device's bearing surfaces.

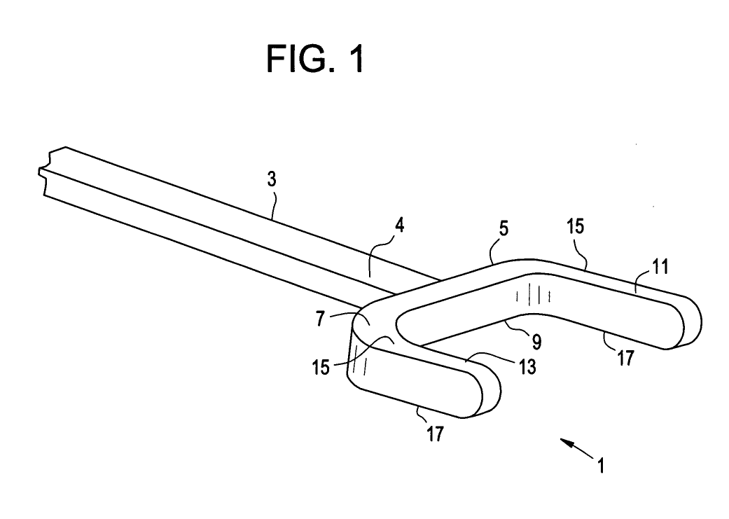

Now referring to FIG. 1, there is provided a seating device 1 comprising:
a) a proximal shaft 3 having a distal end portion 4,
b) an intermediate portion 5 extending transversely from the distal end portion of the proximal shaft and having an upper surface 7 and a lower surface 9, and
c) first 11 and second 13 distal tynes extending distally from the intermediate portion, each tyne having an upper surface 15 and a lower surface 17.

In some preferred embodiments, there is provided a method for seating endplates of an intervertebral disc between upper and lower vertebral endplates, comprising the steps of:
a. providing an upper prosthetic endplate having an outer surface having teeth for engaging the upper vertebral endplate and an inner surface having a peripheral rim,
b. providing a lower prosthetic endplate having an outer surface having teeth for engaging the lower vertebral endplate and an inner surface having a peripheral rim,
c. engaging each prosthetic endplate to its respective vertebral endplate to define a gap between the inner surfaces of the endplates,
d. inserting a seating device comprising a head portion having a thickness less than the gap between the endplates,
e. pivoting the head portion upon an endplate to force the teeth of the prosthetic endplates into the respective vertebral endplates.

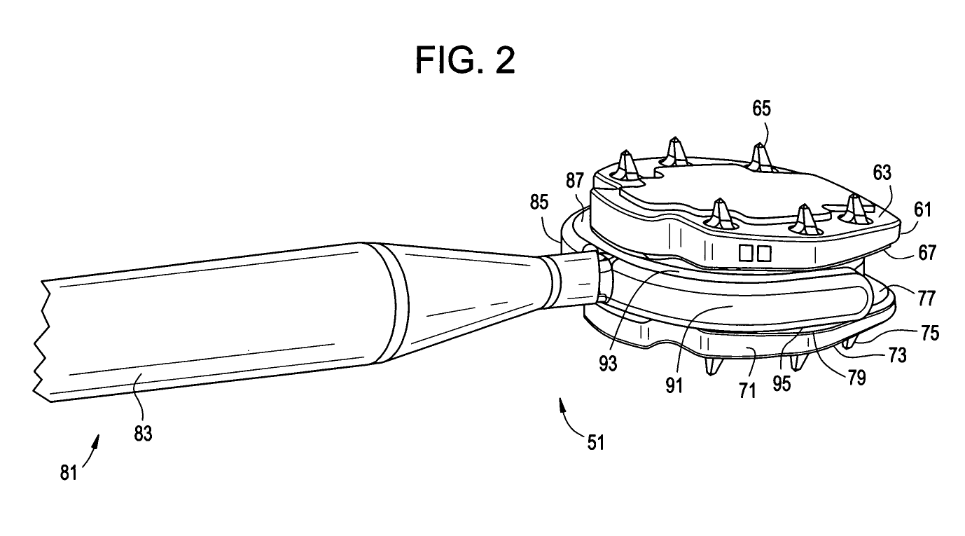

The resultant assembly that results from the above method is shown in FIG. 2. Now referring to FIG. 2, there is provided an assembly 51 for seating prosthetic endplates of an intervertebral disc between upper and lower vertebral endplates, comprising:
a) an upper prosthetic endplate 61 having an outer surface 63 having teeth 65 for engaging the upper vertebral endplate and an inner surface 67 having a peripheral rim (not shown),
b) a lower prosthetic endplate 71 having an outer surface 73 having teeth 75 for engaging the lower vertebral endplate and an inner surface 77 having a peripheral rim 79,
c) a seating device 81 having a proximal shaft 83, an intermediate portion 85 having an upper surface 87, and first (not shown) and second 91 distal tynes extending distally from the intermediate portion, each tyne having an upper surface 93 and a lower surface 95,
   wherein the upper surfaces of the tynes of the seating device bear against the peripheral rim of the upper prosthetic endplate, and
   wherein the lower surfaces of the tynes of the seating device bear against the peripheral rim of the lower prosthetic endplate.

Upon superior or inferior movement of the proximal shaft, a lever is created by which the dovetail becomes a fulcrum and the distal end of the seating device applies a force opposite the movement of the handle. Repeated back and forth motion of the handle applies this levered force to the prosthetic endplates, which results in a seating of the teeth.

The seating device of the present invention enables the surgeon to insert a slim instrument into the disc space and apply a precision force perpendicular to the teeth that will assist in properly seating the prosthetic endplates. The seating device of the present invention does not require an actuation mechanism to apply the seating force and instead uses the mechanical advantage provided by a simple lever.

The seating device of the present invention is designed such that it provides both tactile feedback as well as visual confirmation of accurate placement. Both of these beneficial qualities could greatly reduce the need for additional fluoroscopic imaging that is currently required to confirm seating.

In some embodiments, the intermediate section of the seating device extends transverse to the proximal shaft, and the distal tynes extend from the ends of the transverse intermediate section. The U-shaped head formed by this arrangement allows the seating device to be inserted into the disc space and avoid contact with a core component that has been placed between the prosthetic endplates.

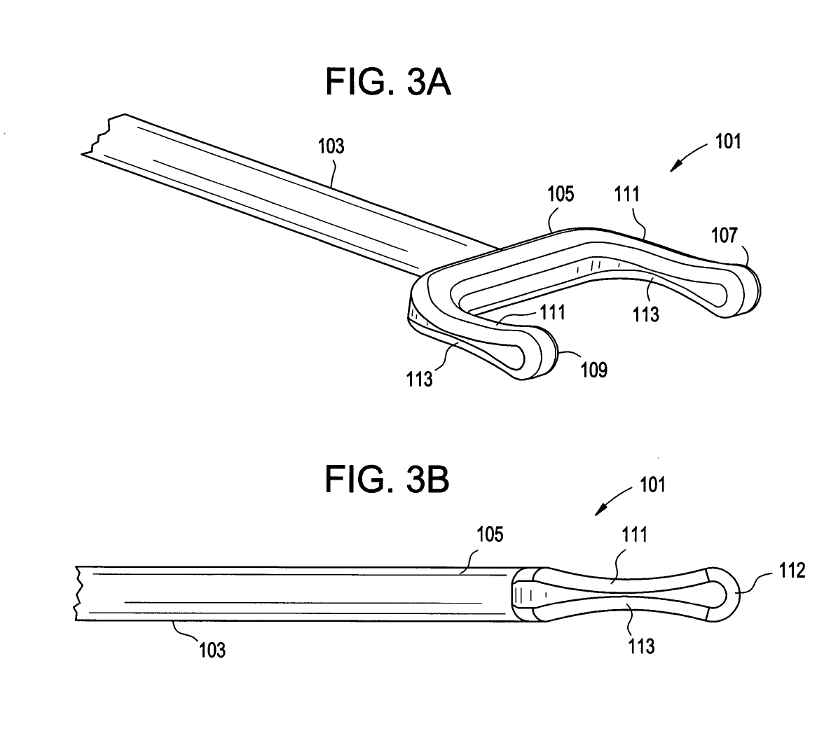

Now referring to FIG. 3b, in some embodiments, the distal ends 112 of the distal tynes are rounded. These rounded ends allow the seating device to smoothly enter gaps which are slightly smaller than the thickness of the distal tynes and gently push the prosthetic endplates apart.

The instrument disclosed herein may be suitably manufactured from any suitable biomaterial, including metals such as titanium alloys, chromium-cobalt alloys and stainless steel), ceramics (such as alumina and zirconia, and mixtures thereof) and polymers (such as PEEK, carbon fiber-polymer composites and UHMWPE).

In some embodiments, at least the upper and lower surfaces of the tynes are coated with a polymer. The polymer coating protects the peripheral rims of the prosthetic endplates from any unwanted abrasion that could occur during the spreading of the endplates by the tynes. In some embodiments, the transverse intermediate section is also coated with the polymer. In some embodiments, the proximal shaft is also coated with the polymer.

In some embodiments, the tynes are contoured with a protrusion to provide point contact with the inner surface of the respective endplate. Preferably, the contour is provided as a concave or convex upper and/or lower surface. Therefore, and now referring to FIGS. 3a and 3b, there is provided a seating device 101 for seating endplates of an intervertebral disc between upper and lower vertebral endplates, comprising:
a) a proximal shaft 103,
b) an intermediate portion 105 extending transverse to the proximal shaft, and
c) first 107 and second 109 distal tynes extending distally from the intermediate portion, each tyne having an upper surface 111 and a lower surface 113,
   wherein at least one of the upper and lower surfaces is contoured with a protrusion to provide point contact with an inner surface of the respective endplate.

In some embodiments, torsional movement of the proximal portion of the device is used to seat the prosthetic endplates. By moving the proximal shaft radial in a first direction, the device will pivot about a first tyne upon a first endplate, and the second tyne will bear against the second prosthetic endplate, thereby causing the prosthetic endplates to separate from each other and pushing the exterior teeth into the bony vertebral endplates. This process can then be repeated by moving the proximal portion of the device in the opposite radial direction, so that the device will pivot about its second tyne upon the second endplate, and the first tyne will bear against the first prosthetic endplate.

In some embodiments, the device of the present invention is intended to be used in conjunction with a distractor that has been seated with caspar pins. However, the presence of the caspar pins (or the distractor tubes that overlie the pins) may interfere with the ability of the device of the present invention to fully pivot. Therefore, in some embodiments of the present invention, a portion of the shaft of the device is offset in order to avoid the caspar pins and/or distractor tubes during operation.

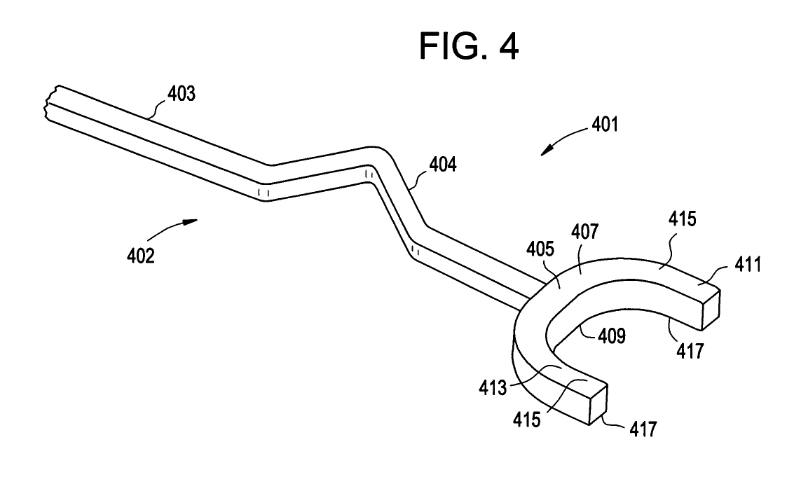

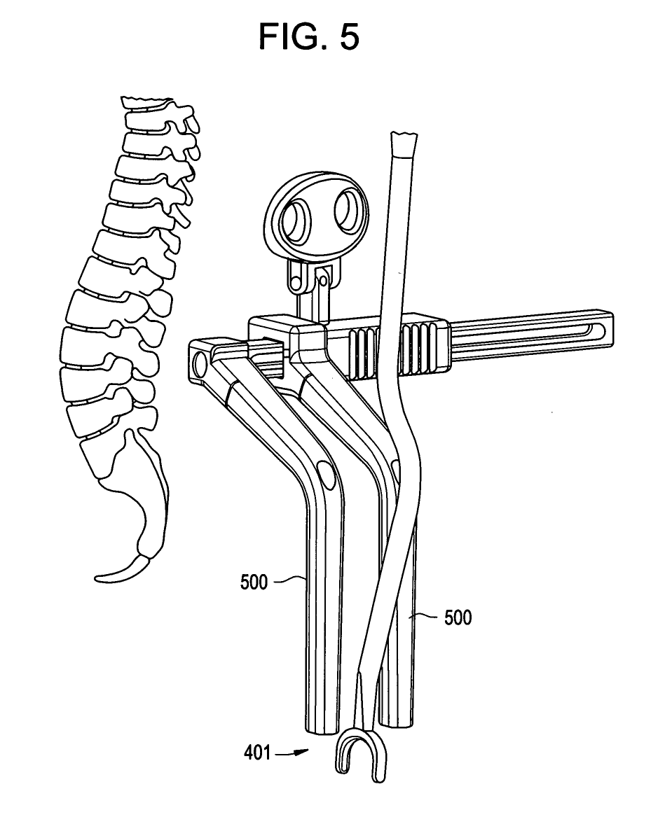

In particular, and now referring to FIG. 4, there is provided a seating device 401 comprising:
a) a proximal shaft 402 having a proximal portion 403 defining a longitudinal axis and a distal end portion 404 offset from the longitudinal axis,
b) an intermediate portion 405 extending transversely from the distal end portion of the proximal shaft and having an upper surface 407 and a lower surface 409, and
c) first 411 and second 413 distal tynes extending distally from the intermediate portion, each tyne having an upper surface 415 and a lower surface 417.
   FIG. 5 shows the device 401 having the offset shaft interposed between a pair of distractor tubes, whereby the offset shaft allows the device to freely pivot without interference from the distractor tubes 500.

In some embodiments, the proximal shaft of the present invention can be modified to include a docking arm. This docking arm can dock to a nearby structure (such as the above mentioned distractor arm) and provide a fulcrum upon which the device of the present invention can pivot. By using the docking arm, the fulcrum is moved more proximal to the surgeon (i.e., from the intermediate portion of the device to the proximal shaft), and so the is advantageous because additional leverage is provided, the clinician needs to produce less of an angle in order to achieve the same bias, and the docking is now being achieved off of a more stable surface.

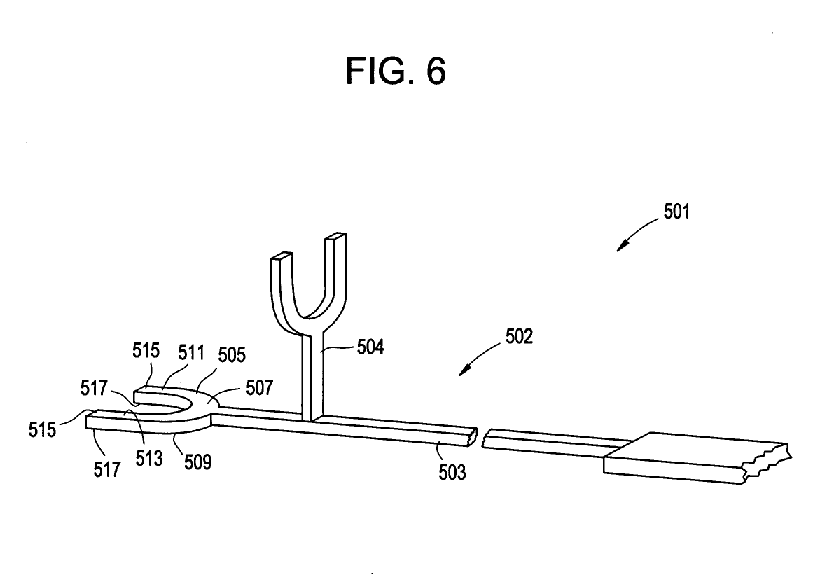

In particular, and now referring to FIG. 6, there is provided a seating device 501 comprising:
a) a proximal shaft 502 having a proximal portion 503 defining a longitudinal axis and a docking arm 504 extending transversely from the longitudinal axis,
b) an intermediate portion 505 extending transversely from the distal end portion of the proximal shaft and having an upper surface 507 and a lower surface 509, and
c) first 511 and second 513 distal tynes extending distally from the intermediate portion, each tyne having an upper surface 515 and a lower surface 517.
   In this particular case, the docking arm 504 has the shape of a tuning fork and is adapted to dock upon an adjacent distractor tube. However, the docking arm can have any other shape that allows it to be stably used as a fulcrum for pivoting the device of the present invention.

In some docking arm embodiments, the device of the present invention has a scissors configuration, wherein the docking arm and head portion are each coupled to pivotally connected shafts.

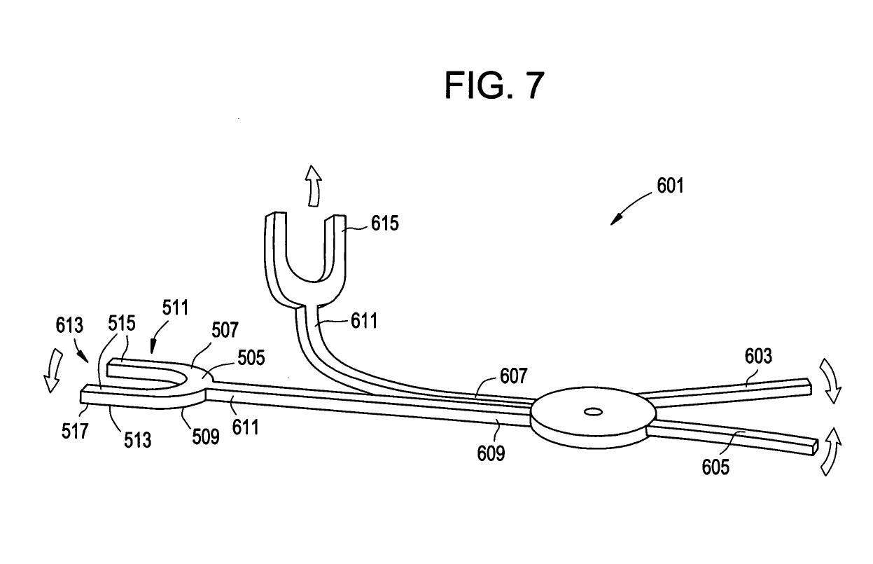

Now referring to FIG. 7, there is provided a seating device 601 comprising:

a) first 603 and second 605 handles defining a first longitudinal axis;

b) first 607 and second 609 shafts respectively pivotally connected with the first and second handles, each shaft having a distal end 611;

c) a head portion 613 for seating prosthetic endplates connected to the distal end of the first shaft, the head portion comprising:

i) an intermediate portion 505 extending transversely from the distal end portion of the proximal shaft and having an upper surface 507 and a lower surface 509, and
ii) first 511 and second 513 distal tynes extending distally from the intermediate portion, each tyne having an upper surface 515 and a lower surface 517, and
   d) a docking arm 615 for docking to a distractor tube connected to the distal end of the second.
   In use, the surgeon slides the device between the distractor tube and docks the docking arm upon one tube while interposing the head portion between the prosthetic endplates. The surgeon then squeezes the handles of the device in order to spread apart of the docking arm and the head, thereby pivoting the head portion upon a prosthetic endplate to force the teeth of the prosthetic endplate into the respective vertebral endplates. The advantages of this design are that additional leverage is provided, the clinician needs to produce less of an angle in order to achieve the same bias, and the docking is now being achieved off of a more stable surface.

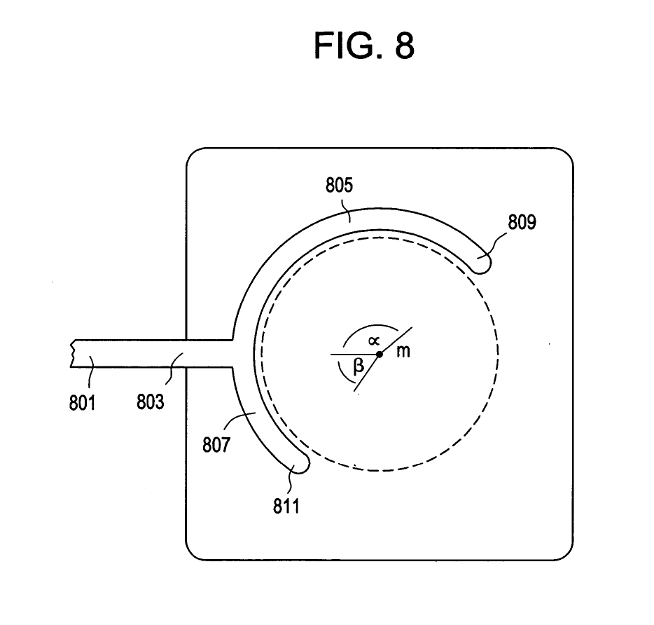

In some embodiments, the tynes are curved to form arcs, and one tyne is shorter than the other tyne. In particular, and now referring to FIG. 8, the first tyne forms an arc describing an angle α of greater than 90 degrees, while the second tyne forms an arc describing an angle β of less than 90 degrees. In this configuration, the device is inserted substantially laterally around a circular articulation surface (dotted lines) so that the end of the longer tyne can contact a posterior portion of the endplate. The ability to contact a posterior portion of the endplate provides an advantage over earlier embodiments in that more full seating of the posterior endplate spikes is now realizable. Once the device seats the posterior endplates as shown in FIG. 8, the device is flipped over 180 degrees in order to seat the posterior endplate spikes on the lower portion of the endplate.

Therefore, in accordance with the present invention, there is provided a seating device for seating endplates of an intervertebral disc between upper and lower vertebral endplates, comprising:
a) a proximal shaft 801 having a distal end 803,
b) first 805 and second 807 tynes extending distally from the distal end of the proximal shaft to form first and second arcs defining a circle having a midpoint “m”, each tyne having an end 809,811,
   wherein the end 809 of the first tyne forms an angle α. with respect to the distal end of the shaft and the midpoint, wherein the angle α is greater than 90 degrees, and.

wherein the end 811 of the second tyne forms an angle β with respect to the distal end of the shaft and the midpoint, wherein the angle β is less than 90 degrees.

Preferably, the first tyne forms an arc describing an angle α of greater than 135 degrees, while the second tyne forms an arc describing an angle β of less than 45 degrees.

Preferably, angle α and angle β total less than 180 degrees, in this condition, the device may be inserted substantially laterally from the side of the endplate.

Preferred articulating motion devices for use with the present invention are disclosed in U.S. Pat. Nos. 5,556,431 and 5,674,296, the specifications of which are incorporated by reference.

In some embodiments, the general structure of the three piece articulating motion disc comprises:

a) a first prosthetic vertebral endplate comprising:
i) an outer surface adapted to mate with a first vertebral body,
   ii) an inner surface having a first articulation surface and a peripheral rim,
   iii) a body portion connecting the inner and outer surfaces,

b) a second prosthetic vertebral endplate comprising:
i) an outer surface adapted to mate with a second vertebral body, and
ii) an inner surface comprising a first articulation surface and a peripheral rim,

c) a core member comprising:
i) a first articulation surface adapted for articulation with the first articulation surface of the first endplate, and
ii) a second articulation surface adapted for articulation with the first articulation surface of the second endplate,
   wherein the core member is oriented to produce a first articulation interface between the first articulation surface of the first endplate and the first articulation surface of the core member, and a second articulation interface between the first articulation surface of the second endplate and the second articulation surface of the core member.

In some embodiments, the general structure of the articulating motion disc is a two piece design and comprises:

a) a first prosthetic vertebral endplate comprising:
i) an outer surface adapted to mate with a first vertebral body,
ii) an inner surface having a first articulation surface and a peripheral rim,
iii) a body portion connecting the inner and outer surfaces,

b) a second prosthetic vertebral endplate comprising:
i) an outer surface adapted to mate with a second vertebral body, and
ii) an inner surface comprising a second articulation surface and a peripheral rim,
   wherein the first and second articulation surfaces are oriented produce an articulation interface.

Preferably, the articulation interfaces form partial spheres.

The motion discs of the present invention can be adapted for use any of the lumbar, thoracic or cervical spine regions. In some embodiments wherein the motion disc is adapted for use in the lumbar region, the three-piece design having a core is selected. In some embodiments wherein the motion disc is adapted for use in the cervical region, the two-piece design is selected.

I claim:

1. An assembly for seating prosthetic endplates of an intervertebral disc between upper and lower vertebral endplates, comprising:
a) an upper prosthetic endplate having an outer surface having teeth for engaging the upper vertebral endplate and an inner surface having a peripheral rim,
b) a lower prosthetic endplate having an outer surface having teeth for engaging the lower vertebral endplate and an inner surface having a peripheral rim,
c) a seating device having a proximal shaft, an intermediate portion, and first and second distal tynes extending distally from the intermediate portion, each tyne having a distal end, an upper surface and a lower surface, wherein the inner surfaces of the endplates oppose each other to define a gap therebetween,
wherein the intermediate portion and the tynes of the seating device are positioned within the gap, and
wherein the thickness of the tynes is less than the thickness of the gap so that the distal end of one tyne does not contact an endplate.

2. The assembly of claim 1 wherein at least one of the inner surfaces of the upper and lower prosthetic endplates comprises an articulation surface.

3. The assembly of claim 1 wherein the inner surfaces of the upper and lower prosthetic endplates each comprise an articulation surface.

4. The device of claim 1 wherein the seating device substantially forms a Y—shape.

5. The assembly of claim 1 wherein the intermediate section extends transverse to the shaft and has an upper surface and a lower surface.

6. The assembly of claim 5 wherein the upper surface of the intermediate portion of the seating device bears against the peripheral rim of the upper prosthetic endplate, and wherein the lower surface of the intermediate section of the seating device bears against the peripheral rim of the lower prosthetic endplate.

7. The device of claim 5 wherein the transverse intermediate section and the tynes substantially form a U-shape.

8. The device of claim 5 wherein the transverse intermediate section has a first thickness and the tynes have a second thickness, wherein the first thickness is greater than the second thickness.

9. The device of claim 1 wherein each tyne has a rounded distal end.

10. The device of claim 1 wherein the upper and lower surfaces of the tynes are coated with a polymer.

11. A seating device for seating endplates of an intervertebral disc between upper and lower vertebral endplates, comprising:
a) a proximal shaft,
b) an intermediate portion extending transverse to the proximal shaft, and
c) first and second distal tynes extending distally from the intermediate portion, each tyne having an upper surface and a lower surface,
wherein at least one of the upper and lower surfaces is contoured with a protrusion to provide point contact with an inner surface of the respective endplate.

12. The device of claim 11 wherein each of the upper and lower surfaces is contoured with a protrusion to provide point contact with an inner surface of the respective endplate.

13. The device of claim 11 wherein at least one of the upper and lower surfaces is concave.

14. The device of claim 11 wherein each of the upper and lower surfaces is concave.

15. The device of claim 11 wherein at least one of the upper and lower surfaces is convex.

16. The device of claim 11 wherein each of the upper and lower surfaces is convex.

17. A method for seating endplates of an intervertebral disc between upper and lower vertebral endplates, comprising the steps of:
a) providing an upper prosthetic endplate having an outer surface having teeth for engaging the upper vertebral endplate and an inner surface having a peripheral rim,
b) providing a lower prosthetic endplate having an outer surface having teeth for engaging the lower vertebral endplate and an inner surface having a peripheral rim,
c) engaging each prosthetic endplate to its respective vertebral endplate to define a gap between the inner surfaces of the endplates,
d) inserting a seating device comprising a head portion having a thickness less than the gap between the endplates, and
e) pivoting the head portion upon an endplate to force the teeth of the prosthetic endplates into the respective vertebral endplates.

18. The method of claim 17 wherein the seating device has a proximal shaft, and the head portion comprises a transverse intermediate portion, and first and second distal tynes extending distally from the intermediate portion, wherein the distal tynes define the thickness of the seating device.

19. The method of claim 17 wherein the seating device has a proximal shaft, and the head portion comprises a transverse intermediate portion and first and second distal tynes extending distally from the intermediate portion, wherein the transverse intermediate portion defines the thickness of the seating device.

20. The method of claim 19 wherein the transverse intermediate portion, and first and second distal tynes of the seating device substantially correspond to the peripheral rims of the prosthetic endplates.

21. The method of claim 19 wherein each tyne has a rounded distal end.

22. The method of claim 19 wherein the transverse intermediate section has a first thickness and the tynes have a second thickness, wherein the first thickness is greater than the second thickness.

23. The method of claim 19 wherein the upper and lower surfaces of the tynes are coated with a polymer.

24. The method of claim 19 wherein at least one of the upper and lower surfaces of the tynes is concave.

25. A seating device for seating a prosthetic vertebral endplate, comprising:
a) a proximal shaft having a proximal portion defining a longitudinal axis and a distal end portion offset from the longitudinal axis,
b) an intermediate portion extending transversely from the distal end portion of the proximal shaft and having an upper surface and a lower surface, and
c) first and second distal tynes extending distally from the intermediate portion, each tyne having an upper surface and a lower surface.

26. A seating device for seating a prosthetic vertebral endplate, comprising:
a) a proximal shaft having a proximal portion defining a longitudinal axis and a docking arm extending transversely from the longitudinal axis,
b) an intermediate portion extending transversely from the distal end portion of the proximal shaft and having an upper surface and a lower surface, and
c) first and second distal tynes extending distally from the intermediate portion, each tyne having an upper surface and a lower surface.

27. A seating device for seating a prosthetic vertebral endplate, comprising:
a) first and second handles defining a first longitudinal axis;
b) first and second shafts respectively pivotally connected with the first and second handles, each shaft having a distal end;
c) a U-shaped head portion for seating prosthetic endplates connected to the distal end of the first shaft,
d) a docking arm for docking to a distractor tube connected to the distal end of the second.

28. The device of claim 27 wherein the head portion comprising:
i) an intermediate portion extending transversely from the distal end portion of the proximal shaft and having an upper surface and a lower surface, and
ii) first and second distal tynes extending distally from the intermediate portion, each tyne having an upper surface and a lower surface.

29. A seating device for seating endplates of an intervertebral disc between upper and lower vertebral endplates, comprising:
a) a proximal shaft having a distal end,
b) first and second distal tynes extending distally from the distal end of the proximal shaft to form first and second arcs defining a circle having a midpoint, each tyne having an end,
wherein the end of the first tyne forms an angle α. with respect to the distal end of the shaft and the midpoint, wherein the angle α is greater than 90 degrees, and.
wherein the end of the second tyne forms an angle β with respect to the distal end of the shaft and the midpoint, wherein the angle β is less than 90 degrees.

30. The device of claim 29 wherein the angle α is greater than 135 degrees.

31. The device of claim 29 wherein the angle β is less than 45 degrees.

32. The device of claim 29 wherein the angle β and angle α total less than 180 degrees.
